(19) United States
(12) Patent Application Publication
(10) Pub. No.: US 2025/0295223 A1
(43) Pub. Date: Sep. 25, 2025

(54) TOOTHBRUSH FOR VERTICAL BRUSHING

(71) Applicant: Chongping LI, Yueyang (CN)

(72) Inventor: Chongping LI, Yueyang (CN)

(21) Appl. No.: 18/882,786

(22) Filed: Sep. 12, 2024

(30) Foreign Application Priority Data
Mar. 22, 2024 (CN) 202420572781.0
Jun. 20, 2024 (CN) 202421423755.8
Jul. 19, 2024 (CN) 202421726451.9

(51) Int. Cl. A46B 5/00; A46B 9/04; A46B 15/00; A61B 17/24

(52) U.S. Cl. CPC A46B 5/0079; A46B 9/04; A46B 15/0081; A61B 17/244; A46B 2200/108

(57) ABSTRACT

The provided is a toothbrush for vertical brushing. The toothbrush includes a handle and a brush head. The brush head is detachably connected to the handle, and the brush head is replaceable, such that the toothbrush is more environmentally friendly. An included angle between a straight line in a length direction of the brush head and a straight line in a length direction of the handle is greater than 90° and smaller than 150°. The handle includes a rotating shaft, the brush head is provided with a shaft hole matched with the rotating shaft, and the handle is capable of rotating by 180° relative to the brush head, such that the handle can be switched between a first position and a second position. The handle is obliquely arranged and can be switched in positions, such that operation is more convenient.

### CROSS-REFERENCE TO THE RELATED APPLICATIONS

This application is based upon and claims priority to Chinese Patent Application No. 202420572781.0, filed on Mar. 22, 2024; Chinese Patent Application No. 202421423755.8, filed on Jun. 20, 2024; and Chinese Patent Application No. 202421726451.9, filed on Jul. 19, 2024, the entire contents of which are incorporated herein by reference.

### TECHNICAL FIELD

The present disclosure relates to the technical field of toothbrushes, and particularly relates to a toothbrush for vertical brushing.

### BACKGROUND

According to advices of dentists and the common sense of brushing teeth, vertical teeth brushing is superior to horizontal teeth brushing. Through the vertical teeth brushing, foreign bodies can be removed from gaps between teeth. The vertical teeth brushing is good for teeth without damage to the teeth, and is more convenient to implement.

In the related art, a brush head and a handle of a toothbrush for vertical brushing are arranged to be perpendicular to each other, which is inconvenient to operate. Moreover, the brush head is fixedly connected to the handle, and the brush head is not replaceable.

### SUMMARY

An objective of the present disclosure is to provide a toothbrush for vertical brushing. A brush head is replaceable, such that the toothbrush is more environmentally friendly. A handle is obliquely arranged, such that operation is more convenient.

In order to achieve the objective, the present disclosure uses the following technical solution:

A toothbrush for vertical brushing includes a handle and a brush head. The brush head is detachably connected to the handle.

The brush head includes a main part and bristles. A lower surface of the main part is provided with the bristles.

An included angle between a straight line in a length direction of the brush head and a straight line in a length direction of the handle is greater than 90° and smaller than 150°.

The handle includes a rotating shaft. The brush head is provided with a shaft hole matched with the rotating shaft.

The handle is for rotating by 180° relative to the brush head, such that the handle is capable of being switched between a first position and a second position.

The toothbrush further includes a tongue scraping part. The tongue scraping part is arranged on one side of the main part away from the bristles. Alternatively, the tongue scraping part is arranged at an end of the handle close to the brush head.

In some embodiments, the handle includes a first part, a second part and a third part that are sequentially connected.

The first part includes the rotating shaft and a first body. A length direction of the first body is parallel to the length direction of the brush head. One end of the first body close to the brush head is provided with the rotating shaft.

The second part includes a second body and a third body. One end of the second body is connected to one end of the first body away from the rotating shaft. The other end of the second body is connected to the third body.

An included angle between the first body and the second body is greater than 90° and smaller than 150°.

The third part includes a fourth body. The third body is connected between the second body and the fourth body. An included angle between the second body and the third body is set as an obtuse angle.

A length direction of the fourth body is parallel to a length direction of the second body.

In some embodiments, the third part further includes a sleeve. A cross section of the fourth body is flat-shaped or oval-shaped. The sleeve is provided with a slot matched with the fourth body.

In some embodiments, a first side surface of the main part is provided with a second recess. When the handle needs to be switched between the first position and the second position, the brush head is fixed by biting the second recess with teeth, such that the handle is rotated for position switching.

In some embodiments, a cross section of the second recess is V-shaped.

In some embodiments, the rotating shaft is provided with cutting bodies, and the cutting bodies are symmetrically arranged, such that a reduced zone is formed. The shaft hole is provided with an opening. The reduced zone passes through the opening, such that the rotating shaft is assembled in the shaft hole.

In some embodiments, the toothbrush further includes a limiting part. The limiting part includes first convex bodies and first concave bodies.

An outer circumferential surface of the rotating shaft is provided with the two first convex bodies. The two first convex bodies are symmetrically arranged.

An inner circumferential surface of the shaft hole is provided with the two first concave bodies. The two first concave bodies are symmetrically arranged.

When the handle is disposed at the first position and the second position, the first convex bodies are connected to the first concave bodies.

In some embodiments, the rotating shaft includes a first shaft section and a second shaft section. A distance is reserved between the first shaft section and the second shaft section.

The first shaft section is spherical. The second shaft section is cylindrical.

The first shaft section and the second shaft section are connected to two ends of the shaft hole respectively.

In some embodiments, the toothbrush further includes a limiting part. The limiting part includes a first convex body and first concave bodies.

The first convex body is arranged on one side of the rotating shaft. A quantity of the first concave bodies is two. The two first concave bodies are symmetrically arranged at two sides of the shaft hole.

When the handle is disposed at the first position and the second position, the first convex body is connected to the two first concave bodies separately.

In some embodiments, a first part of the brush head is provided with a first flange. The main part is provided with a second flange. When the handle is disposed at the first position and the second position, the first flange abuts against the second flange.

The present disclosure has beneficial effects: the brush head is replaceable, such that the toothbrush is more environmentally friendly; the handle is obliquely arranged, such that operation is more convenient; and further the tongue scraping part configured to clean a tongue is arranged to improve functionality.

### BRIEF DESCRIPTION OF THE DRAWINGS

FIG. 1 is an isometric diagram of a toothbrush for vertical brushing according to Example 1 of the present disclosure.

FIG. 2 is a use demonstration diagram of a toothbrush for vertical brushing according to Example 1 of the present disclosure.

FIG. 3 is a top view of a toothbrush for vertical brushing according to Example 1 of the present disclosure.

FIG. 4 is a position switching diagram of a handle according to Example 1 of the present disclosure.

FIG. 5 is an exploded view of a toothbrush for vertical brushing according to Example 1 of the present disclosure.

FIG. 6 is a structural diagram of a handle according to Example 1 of the present disclosure.

FIG. 7 is an exploded view of a handle according to Example 1 of the present disclosure.

FIG. 8 is a side view of a brush head according to Example 1 of the present disclosure.

FIG. 9 is an isometric diagram of a brush head according to Example 1 of the present disclosure.

FIG. 10 is a structural diagram of connection between a brush head and a rotating shaft according to Example 1 of the present disclosure.

FIG. 11 is a structural diagram of a cross section of a shaft hole according to Example 1 of the present disclosure.

FIG. 12 is a structural diagram of a cross section of a rotating shaft according to Example 1 of the present disclosure.

FIG. 13 is a local structural diagram of a brush head and a handle according to Example 2 of the present disclosure.

FIG. 14 is an exploded view of FIG. 13.

FIG. 15 is a local structural diagram of a brush head and a handle according to Example 3 of the present disclosure.

FIG. 16 is an exploded view of FIG. 15.

FIG. 17 is a local structural diagram of a handle according to Example 3 of the present disclosure.

FIG. 18 is a local structural diagram of a brush head and a handle according to Example 4 of the present disclosure.

FIG. 19 is an exploded view of FIG. 18.

FIG. 20 is a local structural diagram of a brush head and a handle according to Example 5 of the present disclosure.

FIG. 21 is an exploded view of FIG. 20.

FIG. 22 is a local structural diagram of a handle according to Example 5 of the present disclosure.

### DETAILED DESCRIPTION OF THE EMBODIMENTS

The present disclosure is further described in detail with reference to the drawings below.

### EXAMPLE 1

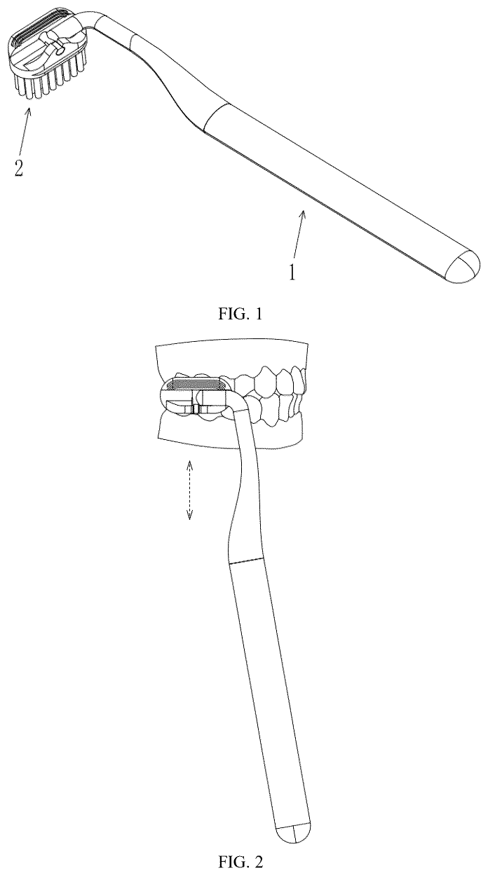

With reference to FIGS. 1-10, a toothbrush for vertical brushing includes a handle 1 and a brush head 2. The brush head 2 is detachably connected to the handle 1, such that both the brush head 2 and the handle 1 are replaceable and reusable, which saves materials and is more environmentally friendly.

The brush head 2 includes a main part 21 and bristles 20. A lower surface of the main part 21 is provided with the bristles 20.

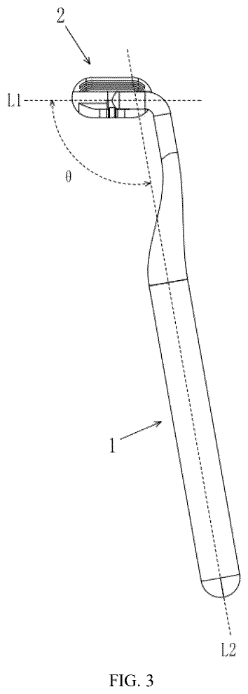

With reference to FIG. 3, an included angle θ between a straight line L1 in a length direction of the brush head 2 and a straight line L2 in a length direction of the handle 1 is greater than 90° and smaller than 150°, such that a vertical brushing structure is formed. Compared with a traditional toothbrush, the toothbrush is smoother to operate and may vertically brush big teeth in an oral cavity more conveniently, such that a cleaning effect can be improved. Preferably, the included angle θ ranges from 95° to 120°, such that operation is smoother and more convenient.

With reference to FIG. 5, the handle 1 includes a rotating shaft 110, and the brush head 2 is provided with a shaft hole 210 matched with the rotating shaft 110, such that relative rotation is implemented.

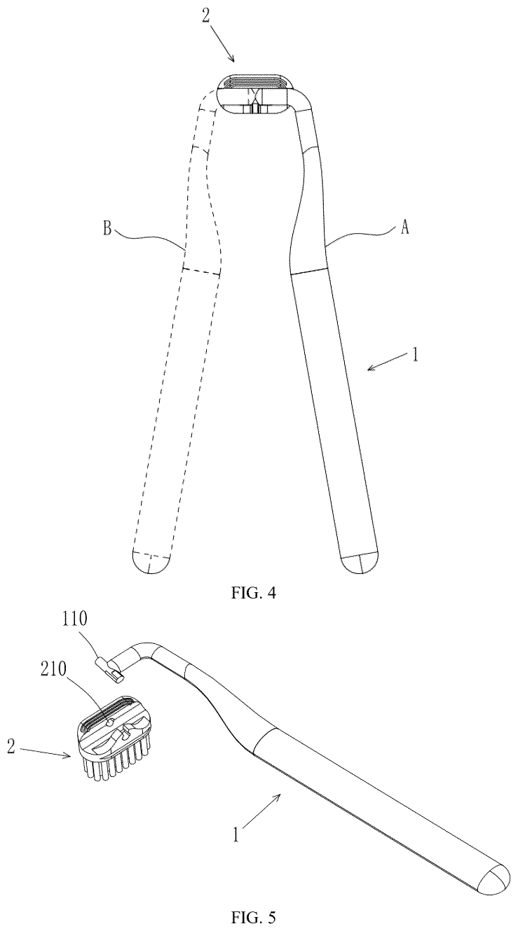

With reference to FIG. 4, the handle 1 is capable of rotating by 180° relative to the brush head 2, such that the handle 1 is capable of being switched between a first position A and a second position B. When the handle 1 is disposed at the first position A, teeth on one side of the oral cavity are cleaned. When the handle 1 is disposed at the second position B, teeth at the other side of the oral cavity are cleaned.

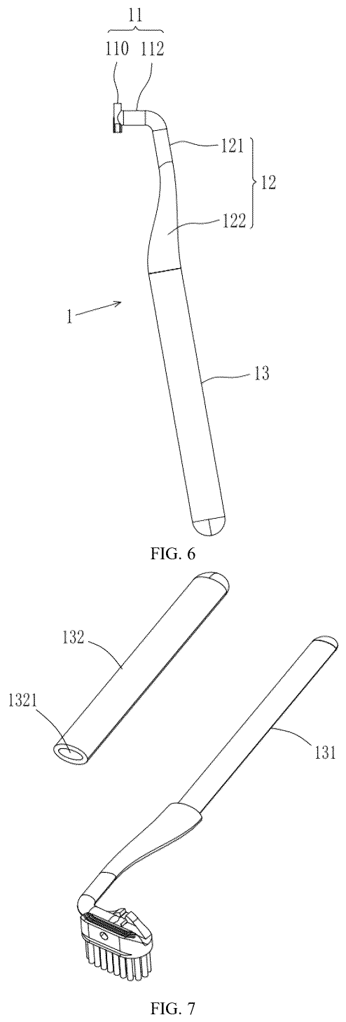

With reference to FIG. 6, the handle 1 includes a first part 11, a second part 12 and a third part 13 that are sequentially connected.

The first part 11 includes the rotating shaft 110 and a first body 112. A length direction of the first body 112 is parallel to the length direction of the brush head 2. One end of the first body 112 close to the brush head 2 is provided with the rotating shaft 110.

The second part 12 includes a second body 121 and a third body 122. One end of the second body 121 is connected to one end of the first body 112 away from the rotating shaft 110. The other end of the second body 121 is connected to the third body 122.

An included angle between the first body 112 and the second body 121 is greater than 90° and smaller than 150°. That is, the included angle equals θ.

The third part 13 includes a fourth body 131. The third body 122 is connected between the second body 121 and the fourth body 131. An included angle between the second body 121 and the third body 122 is set as an obtuse angle. It may be understood that the third body 122 extends obliquely in a direction close to the rotating shaft 110.

A length direction of the fourth body 131 is parallel to a length direction of the second body 121.

The first body 112, the second body 121, the third body 122 and the fourth body 131 may all be cylindrical.

The first part 11 and the second part 12 of the handle 1 define a semi-open zone, which may accommodate a lower jaw or a chin of the oral cavity, such that the brush head 2 may conveniently extend into the oral cavity for cleaning.

The third part 13 of the handle 1 is configured for holding. The third part 13 further includes a sleeve 132. A cross section of the fourth body 131 is flat-shaped or oval-shaped. The sleeve 132 is provided with a slot 1321 matched with the fourth body 131. The sleeve 132 may be made of soft plastic or rubber, such that holding comfort can be improved. Moreover, the sleeve 132 may be disassembled and replaced, which is more environmentally friendly.

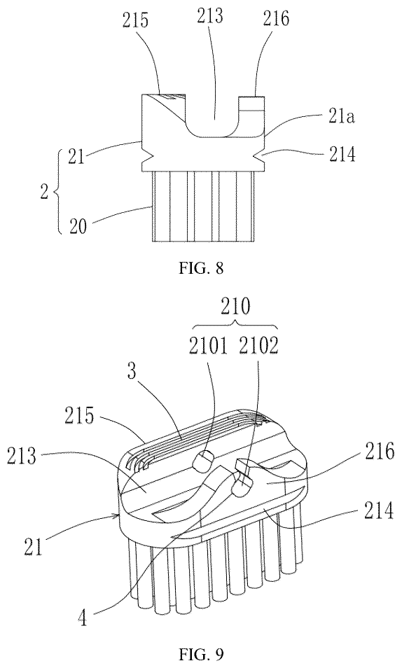

With reference to FIG. 8, a first side surface 21a of the main part 21 is provided with a second recess 214. When the handle 1 needs to be switched between the first position A and the second position B, the brush head 2 is fixed by biting the second recess 214 with teeth, such that the handle 1 is rotated for position switching.

Two second recesses 214 are provided oppositely, and cross sections of the second recesses 214 are V-shaped, such that the second recesses may be engaged with the teeth, and the teeth may bite the brush head 2 more conveniently.

With reference to FIGS. 8-10, an upper surface of the main part 21 of the brush head 2, that is, one side of the main part 21 away from the bristles 20, is provided with a first recess 213, and the first recess 213 is arranged in the length direction of the brush head 2. Then, a first boss 215 and a second boss 216 are formed on two sides of the first recess 213, and the shaft hole 210 penetrates the first boss 215 and the second boss 216. Then, a first hole section 2101 is formed on the first boss 215, a second hole section 2102 is formed on the second boss 216. A first shaft section 1101 of the rotating shaft 110 penetrates the first hole section 2101 and a second shaft section 1102 penetrates the second hole section 2102.

The rotating shaft 110 is provided with cutting bodies 111, and the cutting bodies 111 are symmetrically arranged, such that a section of reduced zone is formed in the rotating shaft 110. The shaft hole 210 is provided with an opening 211. The reduced zone passes through the opening 211, such that the rotating shaft 110 is assembled in the shaft hole 210. Both the first shaft section 1101 and the second shaft section 1102 may be provided with the cutting bodies 111, and the corresponding first hole section 2101 and the corresponding second hole section 2102 are both provided with the openings 211. Preferably, only the second shaft section 1102 is provided with the cutting body 111, and only the second hole section 2102 is provided with the opening 211. During assembly, the first shaft section 1101 is inserted into the first hole section 2101, and then the second shaft section 1102 is assembled in the second hole section 2102 through the opening 211, such that a detachable assembly structure is implemented, and the second shaft section is not easily detached.

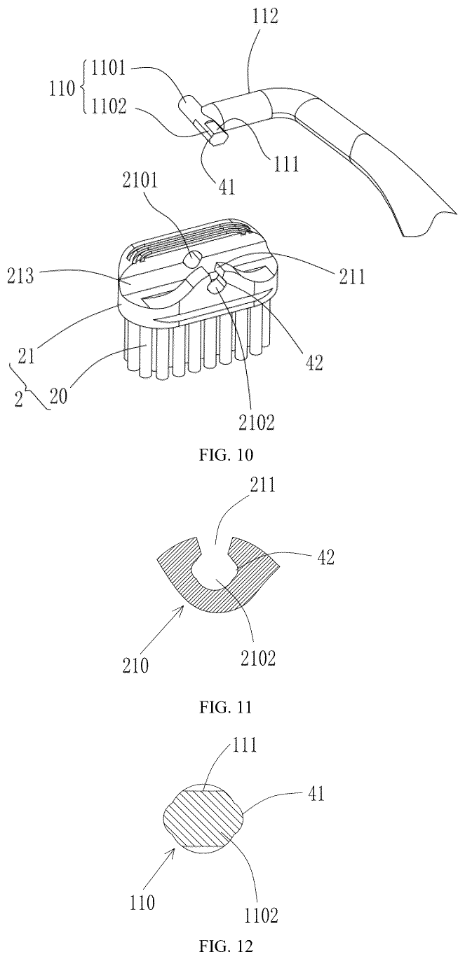

With reference to FIGS. 10-12, the toothbrush further includes a limiting part 4. The limiting part 4 includes first convex bodies 41 and first concave bodies 42.

An outer circumferential surface of the rotating shaft 110 is provided with the two first convex bodies 41. The two first convex bodies 41 are symmetrically arranged.

An inner circumferential surface of the shaft hole 210 is provided with the two first concave bodies 4. The two first concave bodies 4 are symmetrically arranged.

When the handle 1 is disposed at the first position A and the second position B, the first convex bodies 41 are connected to the first concave bodies 4, such that limiting or fixation is implemented. That is, when the handle 1 is disposed at the first position A, one of the first convex bodies 41 is connected to one of the first concave bodies 4. When the handle 1 is disposed at the first position B, one of the first convex bodies 41 is connected to the other first concave body 42.

Both the first shaft section 1101 and the second shaft section 1102 may be provided with the first convex bodies 41, and the corresponding first hole section 2101 and the corresponding second hole section 2102 are both provided with the first concave bodies 4. Preferably, only the second shaft section 1102 is provided with the first concave body 42. In this way, the first shaft section 1101 may be conveniently inserted into the first hole section 2101, and limiting is implemented.

In addition, the first recess 213 may accommodate the first body 112. When the handle 1 is disposed at the first position A and the second position B, the first body 112 abuts against the first recess 213, such that position limiting is further implemented.

With reference to FIG. 9, an upper surface of the first boss 215 is provided with a tongue scraping part 3. The tongue scraping part 3 may be provided with arc-shaped stripes. A tongue may be cleaned by the tongue scraping part 3.

### EXAMPLE 2

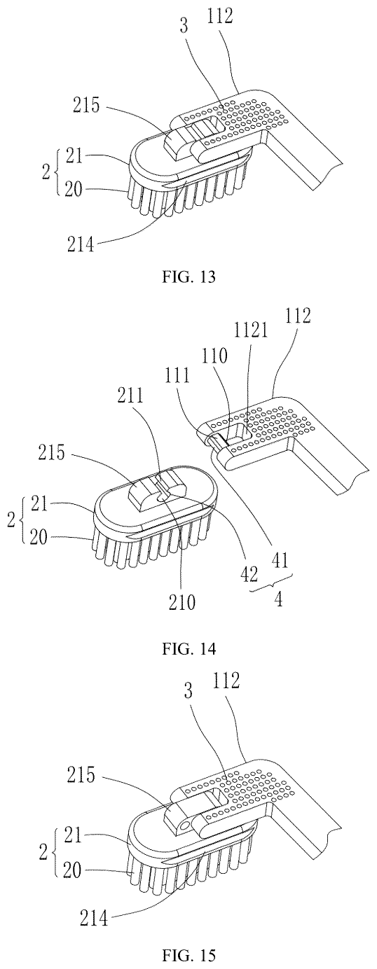

With reference to FIGS. 13 and 14, Example 2 and Example 1 have a basically same structure and are different in that: a top of a main part 21 of a brush head 2 is provided with a first boss 215, the first boss 215 is provided with a shaft hole 210, a first body 112 of a first part 11 of a handle 1 is plate-shaped, the first body 112 is provided with a third recess 1121 capable of accommodating the first boss 215, and the third recess 1121 is provided with a rotating shaft 110.

The rotating shaft 110 is provided with cutting bodies 111. The cutting bodies 111 are symmetrically arranged, such that a section of reduced zone is formed. The shaft hole 210 is provided with an opening 211. The reduced zone passes through the opening 211, such that the rotating shaft 110 is assembled in the shaft hole 210, and detaching and replacement are implemented.

A limiting part 4 includes first convex bodies 41 and first concave bodies 4.

An outer circumferential surface of the rotating shaft 110 is provided with the two first convex bodies 41. The two first convex bodies 41 are symmetrically arranged.

An inner circumferential surface of the shaft hole 210 is provided with the two first concave bodies 4. The two first concave bodies 4 are symmetrically arranged.

When the handle 1 is disposed at a first position A and a second position B, the first convex bodies 41 are connected to the first concave bodies 4, such that limiting or fixation are implemented.

A tongue scraping part 3 is arranged on an upper surface or a lower surface of the first body 112, and alternatively, both an upper surface and a lower surface are provided with tongue scraping parts 3. The tongue scraping part 3 may be stripe-shaped or emboss-shaped.

### EXAMPLE 3

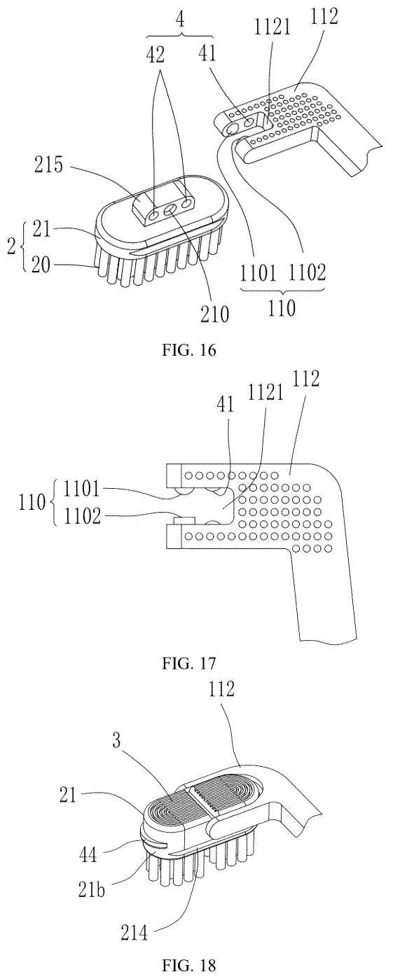

With reference to FIGS. 15-17, Example 3 and Example 2 have a basically same structure and are different in that: a rotating shaft 110 includes a first shaft section 1101 and a second shaft section 1102, and a distance is reserved between the first shaft section 1101 and the second shaft section 1102.

The first shaft section 1101 is spherical. The second shaft section 1102 is cylindrical.

The first shaft section 1101 and the second shaft section 1102 are connected to two ends of a shaft hole 210 respectively. During assembly, the second shaft section 1102 is inserted into one end of the shaft hole 210, and then the first shaft section 1101 is pressed into or pushed into the other end of the shaft hole 210, such that a detachable assembly structure is implemented.

A limiting part 4 includes a first convex body 41 and first concave bodies 4. The first convex body 41 is spherical.

The first convex body 41 is arranged on one side of the rotating shaft 110. For instance, the first convex body 41 is arranged on a recess side wall of a third recess 1121, and the first convex body 41 and the rotating shaft 110 are sequentially arranged in a length direction of a first body 112.

The two first concave bodies 4 are arranged on a same side surface, and the two first concave bodies 4 are symmetrically arranged at two sides of the shaft hole 210. For instance, both the two first concave bodies 4 are arranged on a side surface of a first boss 215, and the shaft hole 210 and the two first concave bodies 4 are disposed at a same horizontal line.

When a handle 1 is disposed at a first position A and a second position B, the corresponding first convex body 41 is connected to the first concave bodies 4, such that limiting is implemented. That is, when the handle 1 is disposed at the first position A, the first convex body 41 is connected to one of the first concave bodies 4. When the handle 1 is disposed at the first position B, the first convex body 41 is connected to the other first concave body 42.

### EXAMPLE 4

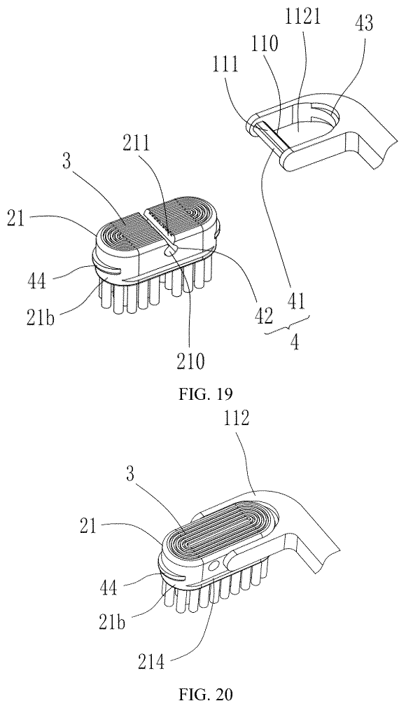

With reference to FIGS. 18-19, Example 4 and Example 1 have a basically same structure and are different in that: a first side surface 21a of a main part 21 of a brush head 2 is provided with a shaft hole 210.

An upper surface of the main part 21 is provided with a tongue scraping part 3. The tongue scraping part 3 is provided with oval-shaped stripes, which is similarly in a track shape.

A first body 112 of a first part 11 of a handle 1 is plate-shaped. The first body 112 is provided with a third recess 1121 capable of accommodating the main part 21. The third recess 1121 is provided with a rotating shaft 110.

The rotating shaft 110 is provided with cutting bodies 111. The cutting bodies 111 are symmetrically arranged, such that a section of reduced zone is formed. The shaft hole 210 is provided with an opening 211. The reduced zone passes through the opening 211, such that the rotating shaft 110 is assembled in the shaft hole 210, and detaching and replacement are implemented.

A limiting part 4 includes first flanges 43 and a second flange 44.

The first flanges 43 may be arranged on a second side surface 21b of the main part 21, and the two first flanges 43 are symmetrically arranged.

The second flange 44 is arranged at an inner side of the third recess 1121.

When the handle 1 is disposed at a first position A and a second position B, first convex bodies 41 are connected to first concave bodies 4 respectively, and the first flanges 43 abut against the second flange 44, such that double limiting is implemented.

### EXAMPLE 5

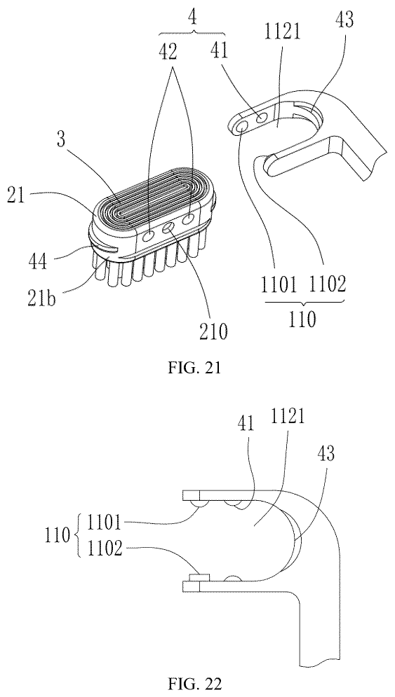

With reference to FIGS. 20-22, Example 5 and Example 4 have a basically same structure and are different in that: a limiting part 4 includes a first flange 43 and a second flange 44, the limiting part 4 further includes a first convex body 41 and first concave bodies 4, and the first convex body 41 is spherical.

The first convex body 41 is arranged on one side of the rotating shaft 110. For instance, a first convex body 41 is arranged on a recess side wall of a third recess 1121, and the first convex body 41 and the rotating shaft 110 are sequentially arranged in a length direction of a first body 112.

The two first concave bodies 4 may be arranged on a same side surface, and the two first concave bodies 4 are symmetrically arranged at two sides of a shaft hole 210. For instance, both the two first concave bodies 4 are arranged on a first side surface 21a of a main part 21, and the shaft hole 210 and the two first concave bodies 4 are disposed at a same horizontal line.

When a handle 1 is disposed at a first position A and a second position B, the first convex body 41 is connected to the first concave bodies 4, and the first flange 43 abuts against the second flange 44, such that double limiting is implemented.

What are disclosed above are merely some embodiments of the present disclosure. Those of ordinary skill in the art can also make several transformations and improvements on the premise of not deviating from the concepts of the present disclosure, and these transformations and improvements shall fall within the protection scope of the present disclosure.

What it claimed is:

1. A toothbrush for vertical brushing, comprising a handle, a brush head, and a tongue scraping part, wherein the brush head is detachably connected to the handle;
the brush head comprises a main part and bristles, and a lower surface of the main part is provided with the bristles;
an included angle θ between a first straight line in a length direction of the brush head and a second straight line in a length direction of the handle is greater than 90° and smaller than 150°;
the handle comprises a rotating shaft, and the brush head is provided with a shaft hole matched with the rotating shaft;
the handle is allowed for rotating by 180° relative to the brush head, wherein the handle is allowed for being switched between a first position and a second position; and
the tongue scraping part is arranged on one side of the main part away from the bristles, or the tongue scraping part is arranged at an end of the handle adjacent to the brush head.

2. The toothbrush for vertical brushing according to claim 1, wherein the handle comprises a first part, a second part and a third part that are sequentially connected;
the first part comprises the rotating shaft and a first body, a length direction of the first body is parallel to the length direction of the brush head, and one end of the first body adjacent to the brush head is provided with the rotating shaft;
the second part comprises a second body and a third body, a first end of the second body is connected to one end of the first body away from the rotating shaft, and a second end of the second body is connected to the third body;
an included angle between the first body and the second body is greater than 90° and smaller than 150°;
the third part comprises a fourth body, the third body is connected between the second body and the fourth body, and an included angle between the second body and the third body is set as an obtuse angle; and
a length direction of the fourth body is parallel to a length direction of the second body.

3. The toothbrush for vertical brushing according to claim 2, wherein the third part further comprises a sleeve, a cross section of the fourth body is flat-shaped or oval-shaped, and the sleeve is provided with a slot matched with the fourth body.

4. The toothbrush for vertical brushing according to claim 1, wherein a first side surface of the main part is provided with a second recess, and when the handle needs to be switched between the first position and the second position, the brush head is fixed by biting the second recess with teeth, wherein the handle is rotated for position switching.

5. The toothbrush for vertical brushing according to claim 4, wherein a cross section of the second recess is V-shaped.

6. The toothbrush for vertical brushing according to claim 1, wherein the rotating shaft is provided with cutting bodies, the cutting bodies are symmetrically arranged, and the shaft hole is provided with an opening.

7. The toothbrush for vertical brushing according to claim 6, further comprising a limiting part, wherein the limiting part comprises first convex bodies and first concave bodies;
an outer circumferential surface of the rotating shaft is provided with the two first convex bodies, and the two first convex bodies are symmetrically arranged;
an inner circumferential surface of the shaft hole is provided with the two first concave bodies, and the two first concave bodies are symmetrically arranged; and
when the handle is disposed at the first position and the second position, the corresponding first convex bodies are connected to the first concave bodies.

8. The toothbrush for vertical brushing according to claim 1, wherein the rotating shaft comprises a first shaft section and a second shaft section, and a distance is reserved between the first shaft section and the second shaft section;
the first shaft section is spherical, and the second shaft section is cylindrical; and
the first shaft section and the second shaft section are connected to two ends of the shaft hole, respectively.

9. The toothbrush for vertical brushing according to claim 8, further comprising a limiting part, wherein the limiting part comprises a first convex body and first concave bodies;
the first convex body is arranged on one side of the rotating shaft;
a quantity of the first concave bodies is two, and the two first concave bodies are symmetrically arranged at two sides of the shaft hole; and
when the handle is disposed at the first position and the second position, the corresponding first convex body is connected to the first concave body.

10. The toothbrush for vertical brushing according to claim 7, wherein a first part of the brush head is provided with first flanges, and the main part is provided with a second flange; and
when the handle is disposed at the first position and the second position, the corresponding first flange abuts against the second flange.
